(19) United States
(12) Patent Application Publication
(10) Pub. No.: US 2015/0265156 A1
(43) Pub. Date: Sep. 24, 2015

(54) OBJECT INFORMATION ACQUIRING APPARATUS AND BREAST EXAMINATION APPARATUS

(71) Applicant: CANON KABUSHIKI KAISHA, Tokyo (JP)

(72) Inventor: Takatoshi Tanaka, Tokyo (JP)

(21) Appl. No.: 14/661,120

(22) Filed: Mar. 18, 2015

(30) Foreign Application Priority Data
Mar. 24, 2014 (JP) 2014-060186

(51) Int. Cl. A61B 5/00; A61B 8/00; A61B 8/14

(52) U.S. Cl. CPC A61B 5/0095; A61B 5/0077; A61B 5/4312; A61B 5/4887; A61B 5/742; A61B 8/14; A61B 8/4416

(57) ABSTRACT

An object information acquiring apparatus is used that includes; a measurement unit comprising a probe that receives acoustic waves from an object, and a position and attitude detector configured to detect at least three positions from among anatomical landmarks of the object; and an information processing device that acquires characteristic information on the interior of the object using the acoustic waves received by the probe.

### BACKGROUND OF THE INVENTION

1. Field of the Invention

The present invention relates to an object information acquiring apparatus and to a breast examination apparatus.

2. Description of the Related Art

Photoacoustic tomography (PAT) apparatuses that rely on the photoacoustic effect have been developed in recent years. In these apparatuses, illumination light (a near infrared ray) from an Nd:YAG laser pulsed light source is irradiated onto an object, acoustic waves generated thereupon inside the object, on account of the photoacoustic effect, are received by electromechanical conversion elements (transducers) in a two-dimensional or three-dimensional array, and an image is generated and displayed. Characteristic information, for instance optical characteristic values, substance concentrations and the like of the object interior, is obtained as a result. This technology is expected to find application, in particular, in diagnostic equipment for breasts and the like.

The specific procedure in photoacoustic effect imaging is as follows.

(1) The positions of two-dimensional array electromechanical conversion elements on the object surface are determined, and single-pulse electromagnetic energy is irradiated onto the object.

(2) Immediately after irradiation of electromagnetic energy, reception signals of the respective electromechanical conversion elements are sampled and recorded.

(3) There is calculated a delay time that it takes an acoustic wave to reach from a point Ps, inside the object to be imaged, up to a position Pt of each electromechanical conversion element i, and signals of the electromechanical conversion elements corresponding to the delay time are added, to yield an image value of the point Ps.

(4) Step (3) above is repeated for each point Ps to be imaged.

Conventionally, measurements in breast diagnostic apparatuses such as photoacoustic measurement apparatuses have involved pinching and holding a breast of a subject lying in a prone position.

In the X-ray mammography apparatus disclosed in Japanese Patent No. 2691073, a breast position at a point in time where a breast is introduced in a breast compression mechanism varies significantly depending on the position and posture of a subject on a table. This apparatus called thus for improvements in terms of securing position reproducibility.

The radiographic imaging system disclosed in Japanese Patent Application Publication No. 2011-177352 failed to address the issue of securing position reproducibility of a same subject over multiple image captures of the latter. Specifically, the system called for further improvements, since position information on the subject as extracted from obtained image information could not be displayed on a radiation irradiating unit.
Patent Literature 1: Japanese Patent No. 2691073
Patent Literature 2: Japanese Patent Application Laid-open No. 2011-177352

### SUMMARY OF THE INVENTION

Breast cancer diagnosis involves in some instance multiple measurements of a breast of a same subject, in order to assess the effect of a prescribed therapeutic agent. Preferably, it should be possible to compare identical segments, from a same direction, in order to verify the effect of the therapeutic agent on the basis of images. However, it is difficult to acquire accurately acoustic images of a same segment, from a same direction, when using the method described in Japanese Patent No. 2691073.

It is a goal of the present invention, arrived at in the light of the above issues, to provide a technology that allows observing and comparing one same segment of an object from a same direction.

To accomplish the object, an aspect of the present invention provides an object information acquiring apparatus, comprising: a measurement unit configured to include a probe configured to receive acoustic waves from an object, and a position and attitude detector configured to detect at least three positions from among anatomical landmarks of the object; and an information processing device configured to acquire characteristic information on the interior of the object, using the acoustic waves received by the probe.

An aspect of the present invention also provides A breast examination apparatus comprising: measurement unit for measuring a breast of a subject; a plurality of cameras configured to capture an image of a body surface of the object; and a position and attitude detector that detects positions of at least a left acromion point, a right acromion point and a most anterior point of the abdomen, from among anatomical landmarks of the subject, using images captured by the plurality of cameras.

The present invention succeeds in providing a technology that allows observing and comparing one same segment of an object from a same direction.

Further features of the present invention will become apparent from the following description of exemplary embodiments with reference to the attached drawings.

### BRIEF DESCRIPTION OF THE DRAWINGS

FIG. 1 is a block diagram of an acoustic measurement apparatus;

FIG. 2 is a perspective view diagram of an acoustic diagnostic apparatus;

FIG. 3A to FIG. 3C are conceptual diagrams of a measurement unit of a first embodiment;

FIG. 4 is an arrangement diagram of anatomical landmarks;

FIG. 5 is a conceptual diagram of coordinate systems of the present invention;

FIG. 6A to FIG. 6C are conceptual diagrams of coordinate conversion using Euler angles;

FIG. 7 is a diagram illustrating an example of display on a display;

FIG. 8A to FIG. 8C are conceptual diagrams of a measurement unit of a second embodiment;

FIG. 9 is a diagram illustrating an example of display on a display of the second embodiment; and

FIG. 10 is a diagram illustrating an example of display on a display of a third embodiment.

### DESCRIPTION OF THE EMBODIMENTS

Preferred embodiments of the present invention will be explained next with reference to accompanying drawings. The dimensions, materials, and shapes of constituent parts, relative positions between the constituent parts, and other features described in the following embodiments are to be modified, as appropriate, in accordance with the configuration of the equipment to which the present invention is to be applied, and in accordance with various other conditions, and do not constitute features that limit the scope of the invention to the disclosure that follows hereafter.

The present invention relates to an apparatus and method for measuring an object, the breast of a subject being herein an appropriate target for such measurement. Accordingly, the present invention can be viewed as a breast examination apparatus and control method thereof, or as a breast examination method. In the case of measurement of the object with acoustic waves, the present invention can be viewed as an acoustic measurement apparatus or control method thereof, or as an acoustic wave measurement method, and can be viewed as an object information acquiring apparatus or control method thereof, or as an object information acquisition method. The present invention can further be viewed as a program for causing the foregoing methods to be executed in an information processing device provided with hardware resources, such as a CPU and the like, and as a storage medium in which such a program is stored. The present invention can also be viewed as an image display method for appropriate display of images with a view to comparing images at a same position and from a same direction.

The object information acquiring apparatus of the present invention includes apparatuses based on photoacoustic tomography, which involves irradiating light (electromagnetic waves) onto an object, and receiving (detecting) propagating acoustic waves that are generated at specific positions inside the object, or at the object surface, on account of the photoacoustic effect. Such object information acquiring apparatuses obtain, for instance in the form of image data, characteristic information of the object interior on the basis of photoacoustic measurements, and, accordingly, are also referred to as photoacoustic apparatuses.

Characteristic information in a photoacoustic apparatus denotes herein the source distribution of acoustic waves that are generated, and an initial sound pressure distribution within the object, resulting from irradiation of light, or the light energy absorption density distribution or absorption coefficient distribution, or concentration distribution of constituent substances in tissues, derived from the initial sound pressure distribution. Constituent substances of tissue include, for instance, blood components such as oxygen saturation distribution, oxidized-reduced hemoglobin concentration distribution, or fat, collagen, water and the like.

The object information acquiring apparatus of the present invention includes ultrasound devices in which acoustic waves are transmitted to an object and reflected waves (echo waves) reflected at specific positions inside the object are received, to obtain thereby characteristic information in the form of image data or the like. Characteristic information in an ultrasound device refers herein to information that reflects morphological information based on reflected waves at locations having dissimilar acoustic impedances in tissue inside the object. The present invention can be used in methods for measuring various information items, pertaining to the breast of a subject, by utilizing on various measurement unit.

The term acoustic wave in the present invention encompasses typically ultrasonic waves, sound waves, and elastic waves referred to as acoustic waves. Acoustic waves generated on account of the photoacoustic effect are referred to as photoacoustic waves or photoultrasonic waves. Electrical signals converted, by a probe, from acoustic waves, are referred to as acoustic signals. Images of the object interior generated on the basis of acoustic signals derived from photoacoustic waves are referred to as photoacoustic images, while images generated on the basis of acoustic signals derived from ultrasonic waves are also referred to as ultrasound images.

### Embodiment 1

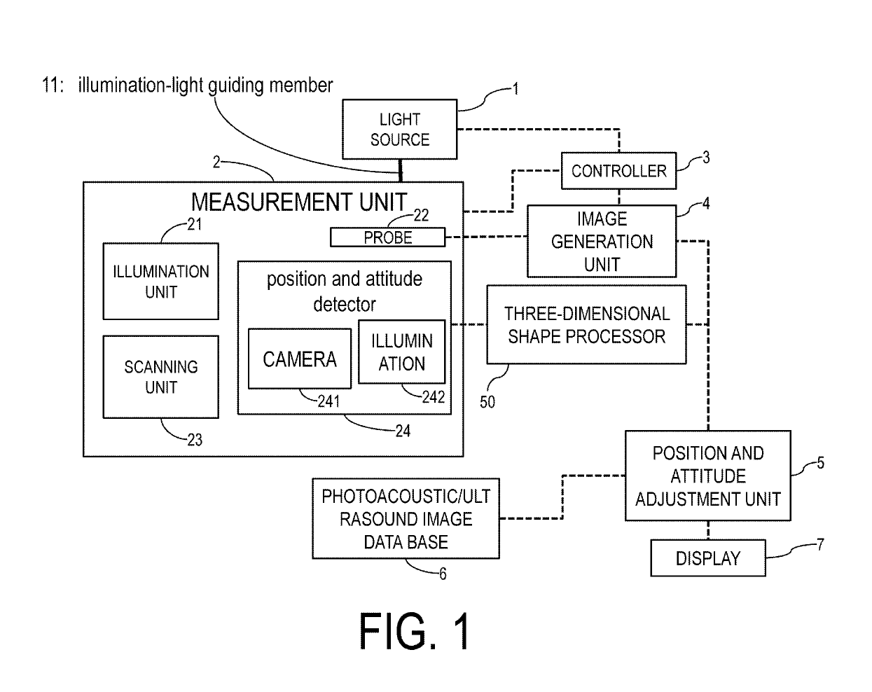

An acoustic measurement apparatus of the present invention will be explained next with reference to FIG. 1. In an acoustic measurement apparatus, illumination light generated in a light source 1 is guided to a measurement unit 2 using illumination-light guiding member 11. Preferably, the position and attitude of the measurement unit 2 can be modified in accordance with the position and posture of a subject. Such a configuration enables imaging at attitudes where little load is exerted on the subject. To achieve this, the illumination-light guiding member 11 is configured such that a light-guiding path, for instance an optical fiber or the like, can be modified flexibly in accordance with the measurement position.

The measurement unit 2 comprises an illumination unit 21, a probe 22, a scanning unit 23 and a position and attitude detector 24. Illumination light that is guided to the measurement unit 2 by the illumination-light guiding member 11 is supplied to the illumination unit 21. The probe 22 comprises two-dimensional array electromechanical conversion elements (transducers) that receive acoustic waves generated on account of the photoacoustic effect, and an ultrasonic probe that transmits and receives ultrasounds. The illumination unit 21 and the probe 22, which are installed on the scanning unit 23, can perform one-dimensional or two-dimensional scanning. The scanning unit 23 may be absent, in the measurement unit 2, in a case where a desired measurement range can be measured without scanning of the probe 22.

The measurement unit 2 comprises the position and attitude detector 24. The position and attitude detector 24, which is made up of cameras 241 and an infrared illumination device 242, detects the positions of anatomical landmarks on the body surface of the subject. As used in the present invention, the term anatomical landmarks denotes landmarks obtained on the basis of the make-up and shape of the human body, and which are characteristic (bulging points, recessed points) of the shape of the body surface and of bones. The position and attitude detector 24 obtains position information, by way of optical unit (cameras 241), from the exterior of the body of the subject.

The position detection method by the position and attitude detector 24 will be explained next. The position and attitude detector 24 of the present embodiment has a plurality of cameras 241. The cameras 241 acquire a two-dimensional image of the body surface of the subject. A three-dimensional shape processor 50 calculates and acquires three-dimensional shape information of the body surface of the subject, on the basis of a plurality of two-dimensional images. The three-dimensional positions of the anatomical landmarks are calculated, on the basis of the three-dimensional shape information, in accordance with image recognition techniques.

If the three-dimensional positions of the anatomical landmarks are to be calculated with yet greater positional precision, an operator arranges markers on the body surface of the subject. The surface of the markers is made up of a material that reflects the infrared light that is irradiated by the infrared illumination device 242. The cameras 241 are infrared cameras that detect infrared light reflected by the marker surfaces. The three-dimensional positions of the markers are calculated by a position and attitude processor 8. The infrared illumination device 242 of the present embodiment is disposed in the measurement unit 2, but, alternatively, markers may be used that emit infrared light. The illumination provided in the position and attitude detector 24 is not limited to infrared illumination. Likewise, the cameras 241 are not limited to infrared cameras. The foregoing may adopt any configuration, so long as an image of light from the illumination device that is provided in the position and attitude detector 24 can be captured by the cameras 241.

The controller 3, which is provided with an electric board (not shown) having a CPU (not shown), electrically controls the various units, light source 1 and the image generation unit 4 inside the measurement unit 2.

The image generation unit 4 generates two-dimensional data and three-dimensional volume data from electrical photoacoustic wave signals and ultrasonic wave signals acquired by the probe 22. Herein, known image reconstruction techniques, such as phasing addition, may be resorted to for data generation. For instance, an information processing circuit or information processing device that operates according to a program can be used as the image generation unit. The image generation unit 4 may share hardware resources, such as an information processing device, storage device and so forth with, for instance, the controller 3 and the below-described position and attitude adjustment unit 5 and three-dimensional shape processor 50, or may be configured separately from these.

The position and attitude adjustment unit 5 calculates the position and posture of the subject of the time at which a three-dimensional photoacoustic image and an ultrasound image are acquired using the three-dimensional positions of the anatomical landmarks as calculated by the three-dimensional shape processor 50. Specifically, the position and attitude adjustment unit 5 calculates a coordinate conversion matrix that denotes the relative positional relationship between a subject coordinate system defined by the anatomical landmarks and a measurement unit coordinate system defined by the measurement unit 2, to calculate thereby relative position and attitude information between the measurement unit 2 and the subject.

A photoacoustic/ultrasound image data base 6 stores, for each subject, photoacoustic images and ultrasound images acquired by measurement, as well as three-dimensional position information calculated by the three-dimensional shape processor 50. Images based on measurement results by some other measurement unit, if any, are also displayed.

The display 7 displays the acquired three-dimensional photoacoustic images and ultrasound images. Herein, three-dimensional images acquired by measurement, as well as three-dimensional images acquired in past measurements, are displayed, with positions and direction matched for the subject, using the coordinate conversion matrix calculated by the position and attitude adjustment unit 5. The display 7 has a monitor for enabling an operator, such as a doctor or technician, to check the adjusted images. Preferably, the display 7 further has election unit for selecting the images to be displayed and selecting the relevant display method.

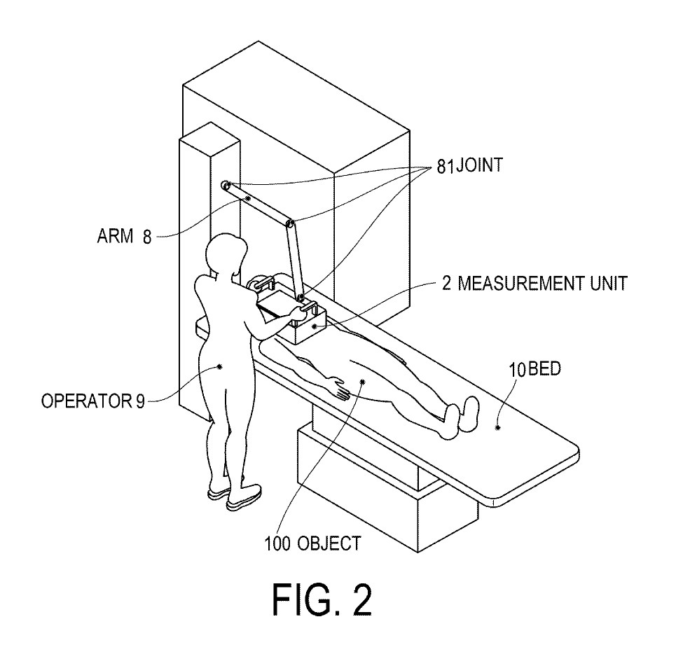

FIG. 2 is a perspective view diagram of a photoacoustic diagnostic apparatus of the present invention. The measurement unit 2 is supported by an arm 8, such that an operator 9 can arrange the measurement unit 2 at a desired position and desired attitude. The operator 9 prompts the object 100 to lie supinely on a bed 10, and carries out measurements while pressing the measurement unit 2 against the chest of the object 100.

The arm 8 has a plurality of joints 81, and further has a lock mechanism (not shown) that locks the measurement unit 2 at the time of measurement. The operator 9 moves the measurement unit 2 to desired positions. Once a position is established, the operator 9 locks the position of the measurement unit 2 using the lock mechanism, and initiates the measurement.

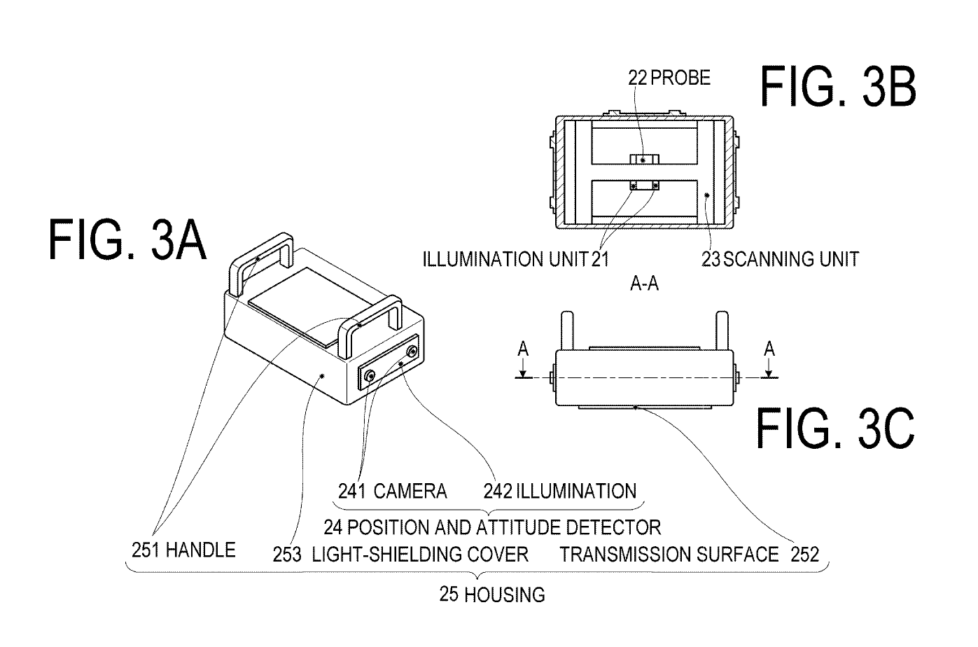

FIG. 3 is a conceptual diagram of the measurement unit 2 of the present invention. FIG. 3A is a perspective view diagram, FIG. 3B is a front-view diagram, and FIG. 3C is an A-A cross-sectional diagram. A housing 25 of the measurement unit 2 comprises handles 251 with which the operator 9 grips the measurement unit 2. The housing 25 is made up of a light-shielding cover 253 that blocks light, and a transmission surface 252 that transmits light and ultrasonic waves. To perform a measurement, the operator 9 grips the handles 251 of the measurement unit 2, presses the transmission surface 252 against the object 100, and locks the measurement unit 2 once the position of the latter has been established. An acoustic matching material such as a gel or the like may be arranged and/or smeared between the transmission surface 252 and the object 100, for the purpose of acoustic impedance matching. The material at the transmission surface 252 may be, for instance, polymethylpentene or the like.

The light-shielding cover 253 has the function of blocking near-infrared light (wavelength 750-1400 nm), in order to protect the operator and the subject from laser light.

Outside the light-shielding cover 253, the measurement unit 2 is provided with the cameras 241 that measure the positions of the anatomical landmarks on the body surface of the object 100. At least two cameras 241 are required in order to grasp the three-dimensional positions of anatomical landmarks. In the present embodiment as well, a plurality of cameras are present outside the light-shielding cover 253. The greater the number of cameras 241 on the light-shielding cover 253, the greater is the detection capability of anatomical landmarks by the position and attitude detector. The cameras 241 are disposed in such a manner that at least three anatomical landmarks can be image-captured from among anatomical landmarks that are present at positions where there is no contact with the transmission surface 252 of the measurement unit 2.

The photoacoustic measurement apparatus of the present invention comprises the cameras 241 in the measurement unit 2. Accordingly, there is a low likelihood that the apparatus structures, or the operator 9, stand between the cameras 241 and the anatomical landmarks, i.e. a state is unlikely to be brought about in which the anatomical landmarks cannot be imaged by the cameras 241. Further, the cameras 241 can be arranged at positions close to the anatomical landmarks, and hence the anatomical landmarks can be detected by the cameras 241 with good positional precision.

The cameras 241 of the present invention are disposed at positions close to anatomical landmarks. Accordingly, the cameras 241 have preferably a wide view angle. The cameras 241 have preferably, therefore, a fish-eye lens as a wide-angle lens. In this case, the three-dimensional shape processor 50 has a function of correcting distortion, caused by using of a fish-eye lens, in the two-dimensional camera image.

The probe 22 has two-dimensional or three-dimensional array electromechanical conversion elements (transducers). In two-dimensional array electromechanical conversion elements, a plurality of piezoelectric elements are disposed two-dimensionally on a same plane. In three-dimensional electromechanical conversion elements, a plurality of piezoelectric elements are disposed on a cylindrical surface or a spherical surface. The view angle of photoacoustic images and ultrasound images is further widened by arranging the elements on a curved surface. The probe 22 further has transmission unit (not shown) for transmitting ultrasonic waves.

The scanning unit 23 has a one-axis or two-axis scanning mechanism. The scanning mechanism is made up of one or a combination of a plurality of electric linear actuators. Thanks to the scanning mechanism, the scanning unit 23 mechanically scans the transducers of the probe 22 one-dimensionally or two-dimensionally. Examples of mechanical scanning methods include, for instance, step & repeat, constant velocity scanning, spiral scanning and the like. The mechanical scanning method is established in accordance with the desired image quality and measurement time.

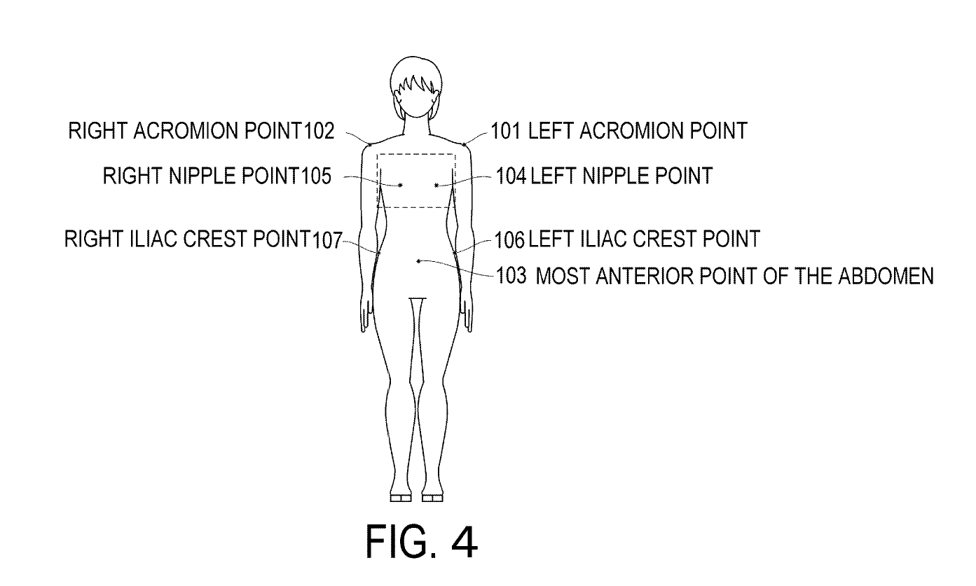

FIG. 4 is an arrangement diagram of anatomical landmarks. Position information on at least three anatomical landmarks is required in order to grasp the relative position an attitude of the measurement unit 2 with respect to the object 100. In a case where the measurement unit 2 of the present invention illustrated in FIG. 2 is used for diagnosis of breast cancer, the operator 9 performs measurements by pressing the measurement unit 2 against the breast. The measurement unit 2 may in some instances measure the entirety of one breast in a single measurement, or may in some instances perform a plurality of measurements of one breast divided into a plurality of segments. A segment to be measured during breast cancer diagnosis lies herein within the dashed line section of FIG. 4.

In the present embodiment, preferably, the conditions below are satisfied by the anatomical landmarks the positions whereof are detected by the cameras 241 that are disposed on the measurement unit 2.

The anatomical landmarks lie outside the segment to be measured (within the dashed line of FIG. 4).

The anatomical landmarks are spaced apart from each other.

The anatomical landmarks are not hidden in the shadow of the body of the object 100, as viewed from the measurement unit 2.

Given the above conditions, a left acromion point 101, a right acromion point 102 and a most anterior point of the abdomen 103 are preferably used as candidates of anatomical landmarks. Other anatomical landmarks may be a left iliac crest point 106 and a right iliac crest point 107, as illustrated in FIG. 4. A left nipple point 104 and a right nipple point 105 are likewise anatomical landmarks, although located within the measurement range of the dashed line.

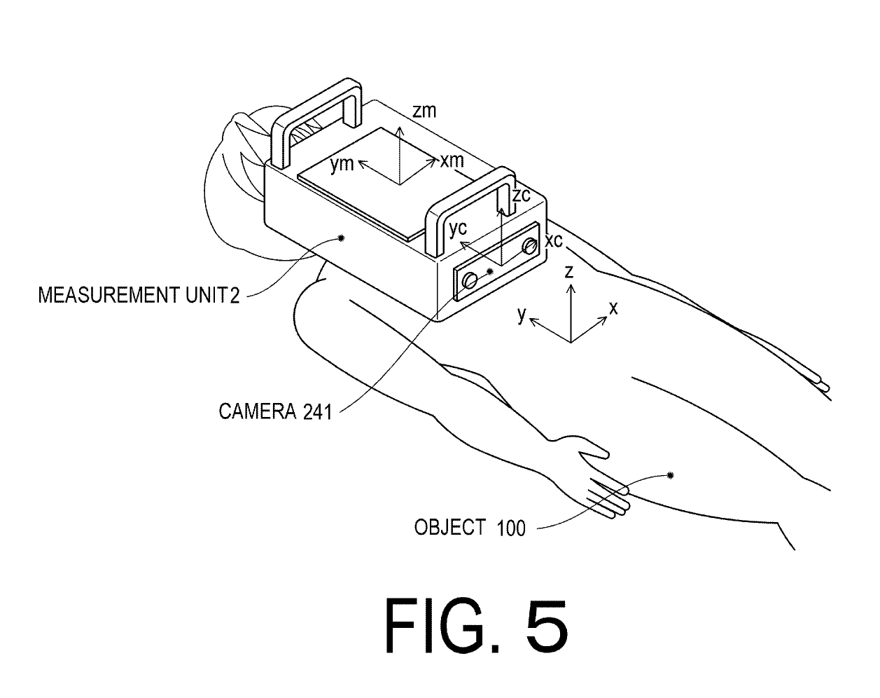

A method for calculating direction cosine matrices in the position and attitude adjustment unit will be explained next with reference to FIG. 5. The measurement unit 2 has a measurement unit coordinate system (xm, ym, zm) and a camera coordinate system (xc, yc, zc). The camera coordinate system is a coordinate system having an imaging sensor of the cameras 241 as the origin. The origin of the measurement unit coordinate system is the center of the acquired three-dimensional photoacoustic image and ultrasound image, in each measurement.

The relative relationships between different measurement unit coordinate systems in each measurement must be calculated in order to match the positions and attitudes of the three-dimensional photoacoustic images and the ultrasound images that are displayed on the display 7. Therefore, the position and attitude adjustment unit 5 calculates direction cosine matrices that denote the relationship between the camera coordinate system, the measurement unit coordinate system and the subject coordinate system, and performs coordinate conversion on the photoacoustic image and the ultrasound image.

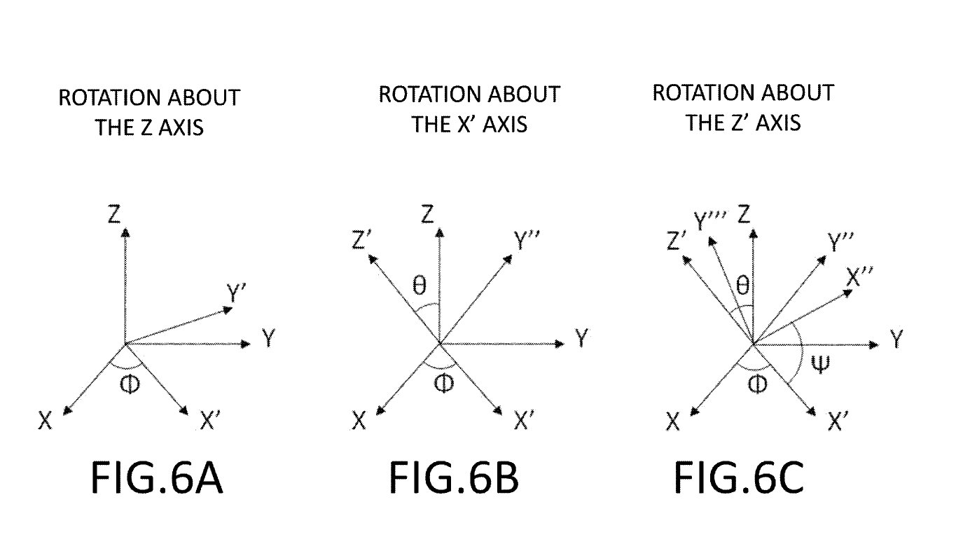

The method for calculating direction cosine matrices using Euler angles will be explained with reference to FIG. 6A to FIG. 6C and the expressions below.

$$[ Math . 1 ]$$

$$\begin{matrix}A = [ \begin{matrix}cos ψ & sin ψ & 0 \\ - sin ψ & cos ψ & 0 \\ 0 & 0 & 1\end{matrix} ] & ( 1 ) \\ B = [ \begin{matrix}1 & 0 & 0 \\ 0 & cos θ & sin θ \\ 0 & - sin θ & cos θ\end{matrix} ] & ( 2 ) \\ C = [ \begin{matrix}cos φ & sin φ & 0 \\ - sin φ & cos φ & 0 \\ 0 & 0 & 1\end{matrix} ] & ( 3 )\end{matrix}$$

In Expressions (1) to (3), each ABC matrix is a respective direction cosine matrix. Conversion from the measurement unit coordinate system to the subject coordinate system, using Euler angles, can be calculated as described above, where A1B1C1 is the direction cosine matrix for the camera coordinate system and the subject coordinate system, and A2B2C2 is the direction cosine matrix for the measurement unit coordinate system and the camera coordinate system.

$$[ Math . 2 ]$$

$$\begin{matrix}[ \begin{matrix}x \\ y \\ z\end{matrix} ] = A 1 B 1 C 1 [ \begin{matrix}xc \\ yc \\ zc\end{matrix} ] & ( 4 ) \\ [ \begin{matrix}xm \\ ym \\ zm\end{matrix} ] = A 2 B 2 C 2 [ \begin{matrix}xc \\ yc \\ zc\end{matrix} ] & ( 5 ) \\ [ \begin{matrix}x \\ y \\ z\end{matrix} ] = {( {( A 1 B 1 C 1 )}^{- 1} )}^{- 1} {( A 2 B 2 C 2 )}^{- 1} [ \begin{matrix}xm \\ ym \\ zm\end{matrix} ] & ( 6 )\end{matrix}$$

The direction cosine matrix of Expression (4) is obtained using the cameras 241. The direction cosine matrix of Expression (5) is already known, as a design value of the measurement unit 2. The position and attitude adjustment unit 5 calculates the attitudes of the three-dimensional photoacoustic image and the ultrasound image in the subject coordinate system using Expression (6). Further, the position of the origin of the measurement unit coordinate system with respect to the subject coordinate system is added, to calculate thereby information, in terms of which is the specific direction and position, with respect to the object 100, of the photoacoustic image and ultrasound image obtained in each measurement.

A coordinate conversion scheme by Euler angles is resorted to in the present embodiment, but the present embodiment is not limited thereto, and coordinate conversion can be accomplished similarly using a quaternion. A singularity problem arises, when using Euler angles, in a case where the measurement unit 2 needs to be rotated significantly with respect to the subject in each measurement, and there exist attitudes for which the direction cosine matrix cannot be calculated. The singularity problem is herein a phenomenon whereby the inverse matrix in Expression (6) cannot be calculated because the denominator is zero. A quaternion is used in such a case. The singularity problem does not arise when using a quaternion.

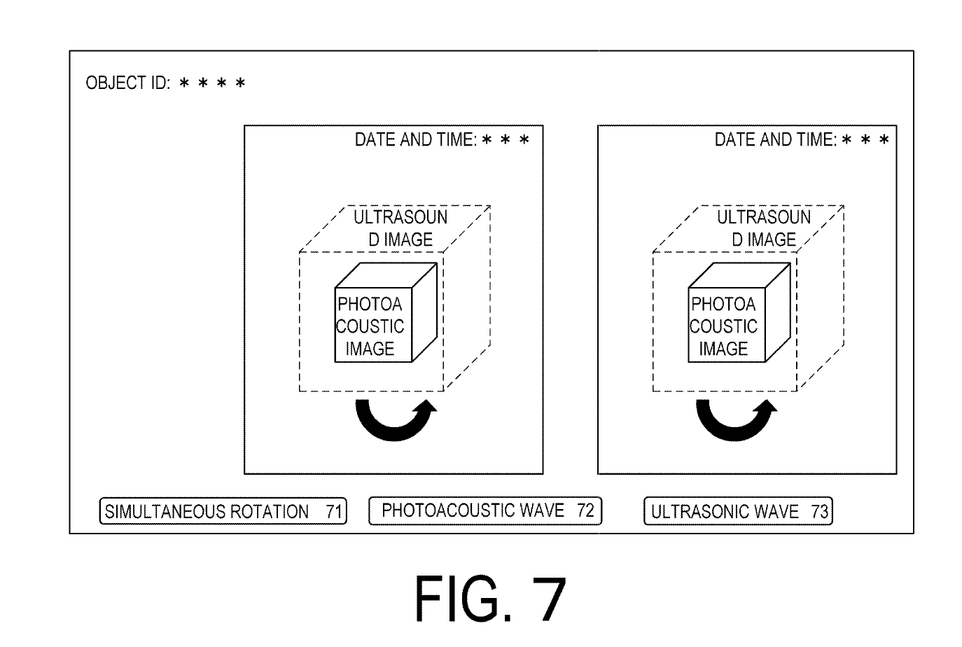

Display of the photoacoustic images and the ultrasound images in the display will be explained next with reference to FIG. 7. The operator and the subject use the display to view and check photoacoustic images and ultrasound images. Photoacoustic images and ultrasound images acquired by the operator over a plurality of measurements are displayed, side by side, on the display of the present invention. The display 7 converts the coordinates of each three-dimensional photoacoustic image and ultrasound image to the subject coordinate system, using the origin position of the measurement unit coordinate system and the direction cosine matrix calculated by the image adjustment unit 5. The display 7 displays then, side by side, three-dimensional photoacoustic images and ultrasound images, of identical coordinate values and identical direction in the subject coordinate system.

If the three-dimensional images are to be displayed in a rotated state, one of the images is rotated by the operator or the subject, through selection of a simultaneous rotation icon 71, as a result of which another displayed image can be likewise rotated automatically. By unselecting the simultaneous rotation icon 71, it becomes then possible to rotate only the image selected for rotation. The operator may use a mouse (not shown), keyboard (not shown) or the like, or may operate a touch panel (not shown) with the fingers, to rotate images and to select/unselect icons.

Photoacoustic images of respective measurements are displayed when a photoacoustic wave icon 72 is selected, and ultrasound images of respective measurements are displayed when an ultrasonic wave icon 73 is selected. Photoacoustic images and ultrasound images are displayed superposed on each other when both icons are selected. The operator can thus recognize changes in image information, visually and in straightforward manner, through display of respective superposed photoacoustic images and ultrasound images, and through matching of positions and attitudes between function information obtained from the photoacoustic images and morphological information obtained from the ultrasound images. The display 7 displays an ID number of the subject. The display 7 displays also the date and time of image-capture for each measurement.

Embodiment 1 of the present invention, as described above, allows comparing a same segment from a same direction in a case where the breast of one same subject is subjected to a plurality of measurements, in order to assess the effect of a prescribed therapeutic agent in diagnosis of breast cancer or the like. The effect of the therapeutic agent can be checked thus on the basis of images. That is, the present invention allows observing and comparing one same segment of an object from a same direction.

### Embodiment 2

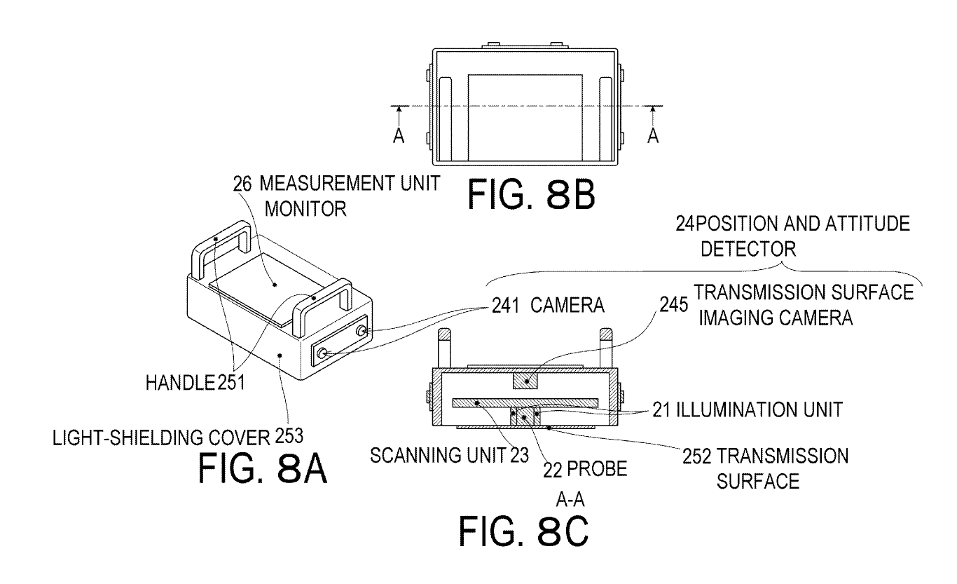

FIG. 8 is a perspective view diagram of a measurement unit of a second embodiment of the acoustic diagnostic apparatus of the present invention. FIG. 8A is a perspective view diagram, FIG. 8B is a bottom-view diagram, and FIG. 8C is an A-A cross-sectional diagram. The measurement unit 2 of the present embodiment comprises the illumination unit 21, the probe 22, the scanning unit 23 and the position and attitude detector 24, as in Embodiment 1. The position and attitude detector 24 of the present embodiment has the cameras 241 outside the light-shielding cover 253. The position and attitude detector 24 of the present embodiment further has, inside the light-shielding cover 253, a transmission surface imaging camera 245 that captures the image of the left and right nipple points of the object 100, via the transmission surface 252. The transmission surface imaging camera 245 detects the positions of the left and right nipple points.

The position and attitude detector 24 of the present embodiment has at least two cameras 241 in order to grasp the three-dimensional positions of anatomical landmarks at positions other than the vicinity of the transmission surface. The cameras 241 are disposed in such a manner that at least three anatomical landmarks can be imaged from among the anatomical landmarks that are present at positions where there is no contact with the transmission surface 252 of the measurement unit 2. The position and attitude detector 24 has at least one transmission surface imaging camera 245 that captures the image of the subject via the transmission surface 252.

The transmission surface imaging camera 245 not only detects the positions of the left and right nipple points, but acquires also a camera image of a contact surface between the transmission surface 252 and the object 100. The operator 9 designates the acquisition area of photoacoustic images and ultrasound images using that camera image. To perform measurements using the photoacoustic measurement apparatus of the present invention, the operator applies or places an acoustic matching material (not shown) between the transmission surface 252 and the object 100. Examples of acoustic matching materials include, for instance, ultrasound gels, gels, water and the like. Acquisition of photoacoustic images and ultrasound images is faulty when bubbles become trapped within the acoustic matching material or at the interfaces between the matching material and the object 100 and the transmission surface 252. Acquisition of photoacoustic images and ultrasound images is likewise faulty when wrinkles form in the matching material or the skin of the object 100. The operator uses the camera image to check the presence or absence of trapped bubbles or interposed wrinkles. If bubbles or wrinkles are found in the image of the transmission surface imaging camera 245, the operator 9 re-adjusts the position and attitude of the measurement unit 2, and then presses the transmission surface 252 against the object 100.

The measurement unit 2 of the present embodiment has a measurement unit monitor 26 in the light-shielding cover 253. The measurement unit monitor 26 displays measured acoustic images, and displays the camera image. Through arrangement of the measurement unit monitor 26 on the light-shielding cover 253, the operator can adjust the position of the measurement unit by gripping the handles 251, while simultaneously bringing the subject and the measurement unit into the field of view.

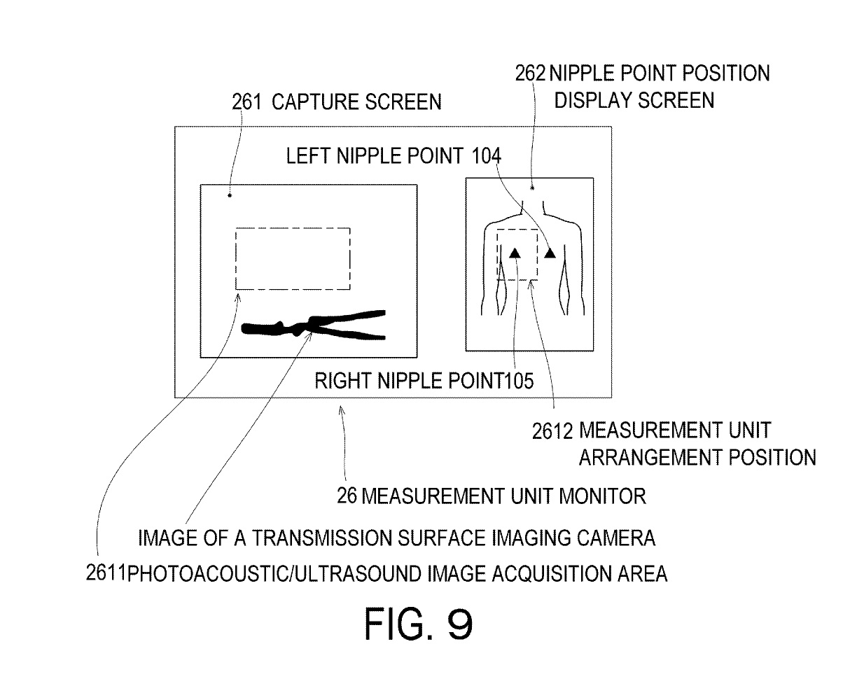

FIG. 9 is a conceptual diagram of a display at the time of display of the transmission surface measurement camera image on the measurement unit monitor 26. A capture screen 261 displays the two-dimensional camera image acquired by the transmission surface imaging camera 245. The operator instructs the area of acquisition of photoacoustic images and ultrasound images on the two-dimensional camera image. A capture screen 261 displays a photoacoustic/ultrasound image acquisition area 2611 instructed by the operator. A nipple point position display screen 262 displays the positions of the left and right nipple points 104, 105 and a measurement unit arrangement position 2612, obtained from computation results of the three-dimensional shape processor, on an picture of the human body. The operator 9 adjusts the position of the measurement unit while checking the capture screen 261 and the nipple point position display screen 262. Specifically, the operator 9 adjusts the position and attitude of the measurement unit 2, while watching the nipple point position display screen 262, in such a manner that the positions of the nipple points in each measurement match each other. By virtue of the above features, the positions of the nipple points, i.e. the position of the breasts, are virtually the same in each measurement.

Once acquisition of photoacoustic images and the ultrasound image is over, the measurement unit monitor 26 displays the three-dimensional photoacoustic images and ultrasound images illustrated in FIG. 7. The operator views and checks the photoacoustic images and the ultrasound images. The measurement unit monitor 26 displays, side by side, the photoacoustic images and ultrasound images acquired by the operator over a plurality of measurements. The display 7 displays the three-dimensional photoacoustic images and the ultrasound image by matching the positions and attitudes of the images that are displayed, using the position information calculated by the image adjustment unit 5. Thanks to such a display scheme, the operator 9 can compare images obtained over a plurality of measurements, and can easily follow up the progress of drug therapy.

To show the images displayed on the measurement unit monitor 26 to the object 100, the operator 9 aims the measurement unit monitor 26 towards the object 100, and show the three-dimensional photoacoustic image and ultrasound image. In the acoustic measurement apparatus of the present invention, the measurement unit 2 is supported by the arm 8 having a plurality of joints 81, as illustrated in FIG. 2. This configuration makes for wider adjustment ranges of the position and attitude of the measurement unit 2, such that the position of the measurement unit monitor 26 can be adjusted according to the posture of the object 100.

Preferably, the measurement unit monitor 26 is of touch panel type. By utilizing a touch panel, the photoacoustic images and ultrasound images that are displayed on the measurement unit monitor 26 are of a large size while in a measurement unit 2 having a compact configuration.

The configuration of the present embodiment allows assisting the operator in adjusting the position of the measurement unit through display, on the measurement unit monitor, of images captured by the transmission surface imaging camera, as described above. The present embodiment as well elicits likewise the effect of making it possible, when implementing breast cancer diagnosis, to compare a same segment from a same direction, and to check the effect of a therapeutic agent on the basis of images, in a case where a breast of a same subject is measured over a plurality of times in order to assess the effect of a prescribed therapeutic agent.

### Embodiment 3

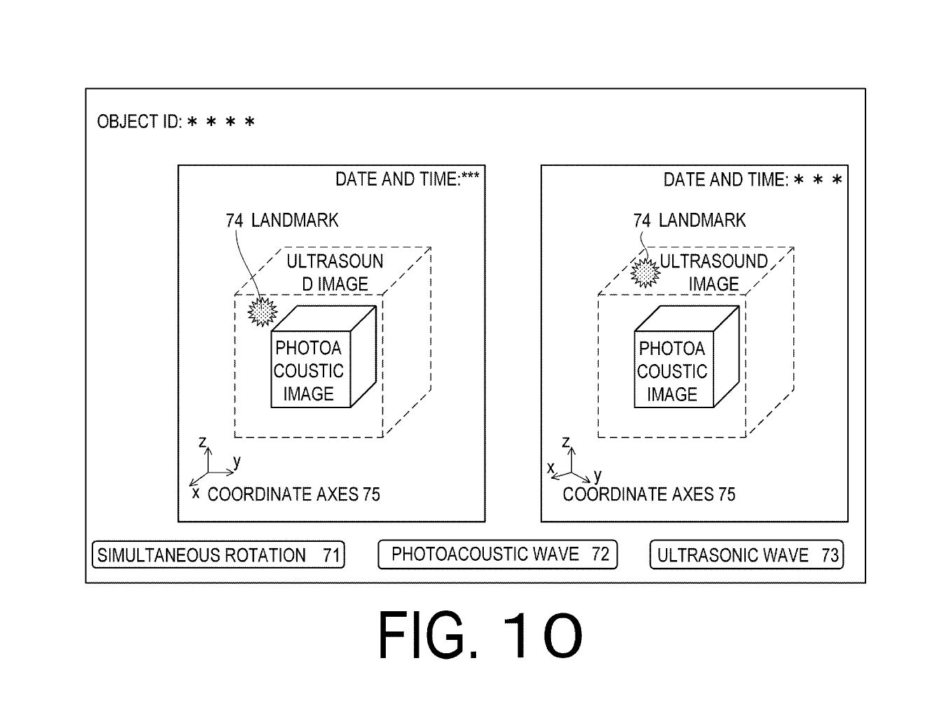

The display of the present embodiment will be explained with reference to FIG. 10. As in the case of Embodiment 1, photoacoustic images and ultrasound images acquired by the operator over a plurality of measurements are displayed, side by side, on the display. In Embodiment 3, moreover, there are displayed also a landmark 74 that denotes the position of an anatomical landmark, and coordinate axes 75 that denote the direction of a three-dimensional image in the subject coordinate system, on each three-dimensional photoacoustic image and ultrasound image. The landmark 74 is at least a mark that denotes the position of either one of the left and right nipple points illustrated in FIG. 4, and is displayed on the three-dimensional photoacoustic image or ultrasound image in such a manner that the landmark 74 is readily recognized by the operator and the subject. The coordinate axes 75 represent directions of respective axes in such a manner that the operator and the subject can recognize directions in the various axes of the subject coordinate system. The coordinate axes 75 are displayed at a position that does not overlap with a three-dimensional volume image.

Thanks to the above configuration, the operator can compare three-dimensional images at a same position and from a same direction, by referring to the landmark 74 and the coordinate axes 75. It becomes therefore easier to notice differences between measurements of the three-dimensional images.

As described above, the technology provided by the present invention allows observing and comparing a same segment of an object from a same direction.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-060186, filed on Mar. 24, 2014, which is hereby incorporated by reference herein in its entirety.

What is claimed is:

1. An object information acquiring apparatus, comprising:
a measurement unit configured to include a probe configured to receive acoustic waves from an object, and a position and attitude detector configured to detect at least three positions from among anatomical landmarks of the object; and
an information processing device configured to acquire characteristic information on the interior of the object, using the acoustic waves received by the probe.

2. The object information acquiring apparatus according to claim 1,
wherein the position and attitude detector has a plurality of cameras configured to capture an image of a body surface of the object.

3. The object information acquiring apparatus according to claim 2, further comprising:
a shape processor configured to detect positions of the anatomical landmarks, using images captured by the plurality of cameras.

4. The object information acquiring apparatus according to claim 2,
wherein the object is a breast; and
a left acromion point, a right acromion point and a most anterior point of the abdomen are used as the anatomical landmarks.

5. The object information acquiring apparatus according to claim 2, further comprising:
an infrared illumination configured to irradiate infrared light, wherein the plurality of cameras are infrared cameras.

6. The object information acquiring apparatus according to claim 2, wherein the plurality of cameras have a fish-eye lens.

7. The object information acquiring apparatus according to claim 2, wherein the plurality of cameras capture an image a marker disposed on the body surface of the object.

8. The object information acquiring apparatus according to claim 2, further comprising a housing of the measurement unit, wherein
the housing has:
a transmission surface which, during reception of acoustic waves by the probe, is pushed against the object and transmits acoustic waves from the object; and
a light-shielding cover, and
the plurality of cameras are disposed outside the light-shielding cover.

9. The object information acquiring apparatus according to claim 8, further comprising:
a light source; and
an illumination unit configured to, inside the light-shielding cover, irradiate light onto the object,
wherein the probe receives photoacoustic waves generated as a result of irradiation of light by the illumination unit.

10. The object information acquiring apparatus according to claim 8, wherein the transmission surface transmits light,
the object information acquiring apparatus further comprising
a transmission surface imaging camera configured to, inside the light-shielding cover, capture an image of the object via the transmission surface.

11. The object information acquiring apparatus according to claim 8, wherein the measurement unit has a measurement unit monitor on the light-shielding cover.

12. The object information acquiring apparatus according to claim 11, wherein the measurement unit monitor displays an image captured by the transmission surface imaging camera, and an image that denotes the position of the measurement unit and positions of left and right nipple points of the object, in an area at which the characteristic information is acquired.

13. The object information acquiring apparatus according to claim 8, wherein the measurement unit is supported by an arm having a plurality of joints.

14. The object information acquiring apparatus according to claim 1, further comprising a scanning unit configured to scan the probe.

15. The object information acquiring apparatus according to claim 1, further comprising a display configured to display an image that represents the characteristic information.

16. The object information acquiring apparatus according to claim 15, wherein the display displays landmarks that denote positions of anatomical landmarks of the object, and displays coordinate axes of the image that represents the characteristic information.

17. A breast examination apparatus comprising:
measurement unit for measuring a breast of a subject;
a plurality of cameras configured to capture an image of a body surface of the object; and
a position and attitude detector that detects positions of at least a left acromion point, a right acromion point and a most anterior point of the abdomen, from among anatomical landmarks of the subject, using images captured by the plurality of cameras.
